(19) United States
(12) Patent Application Publication
(10) Pub. No.: US 2005/0087434 A1
(43) Pub. Date: Apr. 28, 2005

(54) Method and apparatus for transforming chemical fluids using halogen or oxygen in a photo-treatment process

(76) Inventor: Gregorio Tarancon, Punta Gorda, FL (US)

Correspondence Address: EZRA SUTTON, PA;PLAZA 9, 900 ROUTE 9, WOODBRIDGE, NJ, 07095, US

(21) Appl. No.: 10/690,675

(22) Filed: Oct. 22, 2003

(51) Int. Cl. C07C 6/00

(52) U.S. Cl. 204/157.3; 204/158.21

(57) ABSTRACT

A method of treatment of reactant fluids such as hydrochlorofluorocarbons (HCFCs), hydrofluorocarbons (HFCs), hydrochlorocarbons (HCCs), and hydrocarbons (HCs) for the production of new chemical fluids. Another method of treatment for the transformation of the reactant fluids having impurities present in the chlorofluorocarbons (CFCs) or fluorocarbons (FCs) for yielding a high quality chemical product. Reactant fluids with impurities present in used CFC or FC may form an azeotropic mixture. A photochemical reaction is used wherein the reactant fluids are molecules with hydrogen atoms in a hydrogen-carbon bond. The process is comprised of the following steps: placing the reactant fluids into a process compartment of the photochemical reactor; placing halogen fluid or oxygen fluid into the process compartment of the photochemical reactor, wherein the halogen fluid is selected from a group consisting of chlorine (Cl₂), bromine (Br₂) and iodine (I₂); and irradiating the fluids and the halogen or oxygen fluid using radiant energy from lamps operating in the visible and ultraviolet light regions of the electromagnetic spectrum to conduct thermolysis, photolysis and photochemical treatment by halogenating or oxidizing the molecules of the reactant fluids with the halogen or oxygen fluids to form halogenated or oxidized fluids during a dwell time period.

### FIELD OF THE INVENTION

The present invention relates to a photochemical reactor and process where photochemical reaction changes the molecules of fluids containing hydrogen atoms; the hydrogen atoms having a hydrogen-carbon bond in the molecules of the fluid. More particularly, the photochemical reaction process changes the molecules of fluids containing hydrogen that have formed an azeotropic mixture or pseudo-azeotropic mixture with used chloroflourocarbon fluids.

1. Background of the Invention

In 1974, M. J. Molina and F. S. Roland hypothesized that chemicals called chlorofluorocarbons (CFCs) were causing depletion of the ozone layer that protects the earth from harmful levels of ultraviolet radiation, as these fully halogenated CFCs were extremely stable. These halogenated CFCs did not break down in the lower atmosphere or troposphere but remained intact for decades. Eventually, the CFCs made their way to the upper atmosphere where the ultra violet light infringed upon the CFC molecules causing these CFC molecules to decompose and liberate chlorine gas to the upper atmosphere. The chlorine gas then reacts with the ozone layer which then depletes the stratospheric ozone.

Further, the environmental scientific community has been publishing the effect of CFCs to the ozone layer and confirms that the fully halogenate CFCs are extremely stable even under the irradiation of visible and ultraviolet light. Irradiation with radiant energy of visible and ultraviolet light over the elemental chlorine gas divides the molecules of chlorine into atom radicals. The molecules that contain hydrogen atoms react with the chlorine radicals and substitute the hydrogen atoms by chlorine and form chlorinated impurities and hydrogen chloride.

The U.S. Environmental Protection Agency (EPA) for the regulation of CFCs became effective in the early 1990's and major production of CFCs stopped. Alternatives for CFCs were found for many chemical applications but in some cases the use of CFCs is still required. One of the CFCs where there still is no alternative chemical substitute for certain types of applications is the chemical 1,1,2 trichloro 1,2,2 trifluoro-ethane (CFC-113). The only practical option is to recycle and reuse the CFC-113 fluid.

List of chemical fluids and potential contaminants with a boiling point approaching the boiling point of CFC-113:

| | |
|---|---|
| Acetylene dichloride | Difluorobromopropylene |
| Cycle pentane | Hefluorochlorobutane |
| Neo hexane | Tetrafluorodibromoethane |
| Propyl chloride | Heptafluorodimethyloctanedione |
| Trifluorochloroeathane | Heptafluoropropyl-tetrafluoroethyl-ether |
| Trifluorodichloroethane | Perfluoro-tert-butanol |
| Trifluorobromochloroethane | Hexane |
| Difluorodichloroethane | Methylene chloride |
| Fluorochloroethane | Methyl chloride |
| Difluoroethane | Pentane |
| Pentafluorodichloropropane | Carbon disulphide |
| Difluorobromothane | Dimethyl-zinc |

Family of Chemical Fluids with Similar Boiling Points:
Octafluorocylebutane
Hexafluoro-1,3-butadiene
Hexafluorocyclebutene
Perfluorobutene
Perfluoroisobutane
Hexafluoropropane.

In the process of purification by distillation, when impurities are fluids with molecules containing hydrogen and the boiling point of those fluids are approaching the boiling point of the used CFC-113 or when the composition of the liquid mixture and the composition of the vapor mixture are the same, an azeotropic condition occurs, and the distillation process is incapable of purifying to the specified requirement. The foregoing purification techniques of distillation, adsorption or extraction are inadequate to meet purity specifications of 99.99% with respect to total impurities, with a hydrocarbon concentration of less than 1 ppm; a moisture content of less than 5 ppm; and having non-detectible solids therein. The above purity specification corresponds to a virgin CFC-113 product. If the mixture of CFC and contaminant fluids having hydrogen contained in their molecules is treated in the photochemical reactor of this invention, any azeotropic condition disappears and the polarity and solubility changes. The standard process techniques of physical separation (i.e., distillation) can be employed so that the CFC can be purified to the desired specifications.

Additionally, the photochemical process should include halogenation or oxidation of the contaminant fluid by irradiation with UV light, such that all of the impurities in the used CFC-113 fluid can be chlorinated or oxidized and easily separated from the CFC-113 fluid by standard purification techniques. Further, the photochemical treatment process should use a shell and tube-lamp photochemical reactor (detailed in this document) for the transformation of the chemical impurities in the used CFC-113 fluid.

2. Description of the Prior Art

Prior art patents which relate to this technology include U.S. Pat. No. 3,968,178 to Obrecht et al; U.S. Pat. No. 3,993,550 to Deno et al; U.S. Pat. No. 4,043,886 to Bierker et al; U.S. Pat. No. 4,456,512 to Bieler et al; U.S. Pat. No. 5,484,932 to Marhold; and U.S. Pat. No. 6,126,095 to Matheson et al.

None of the prior art references disclose or teach a photochemical reaction for changing an azeotropic condition to a non-azeotropic condition. Further, the prior art does not disclose or teach halogenation or oxidation using a photochemical reaction to change the chemical impurities which are normally not separable by physical means. Additionally, the prior art does not disclose or teach a process for removing azeotropic conditions from the mixture of fluid impurities and CFC's.

Accordingly, it is an object of the present invention to provide a photochemical reactor, in the form of a shell and tube-lamp reactor, such that the tube-lamp therein irradiates radiant energy of visible and ultraviolet light in the electromagnetic spectrum in order to halogenate and/or oxidize the impurities contained in the used CFC-113 fluid.

Another object of the present invention is to provide a photochemical reactor in order to halogenate or oxidize the chemical fluids and other contaminated fluids, in the form of azeotropic and/or pseudo-azeotropic mixtures, within the used CFCs, such that the photochemical reaction transforms the chemical impurities which then changes the physical and chemical properties of the contaminants and CFC mixtures and all of the azeotropic conditions disappear.

Another object of the present invention is to provide a photochemical reactor that uses radiant tube-lamps in the irradiation process of radiating heat and energy using visible and ultraviolet light in order to promote the thermolysis and photolysis of molecules, such as chlorine (Cl₂) and oxygen (O₂) molecules.

Another object of the present invention is to provide a photochemical reactor that is capable of transforming impurities from mixtures of used chlorofluorocarbons (CFCs) and fluorocarbons (FCs).

Another object of the present invention is to provide a photochemical reactor for the chlorination or oxidation of hydrochloflourocarbons (HCFC's) of an azeotropic mixture with chloroflourocarbons (CFC's).

Another object of the present invention is to provide a photochemical reactor for the chlorination or oxidation of hydroflourocarbons (HFC's) of an azeotropic mixture with chloroflourocarbons (CFC's).

Another object of the present invention is to provide a photochemical reactor for the chlorination or oxidation of hydrochlorocarbons (HCC's) of a mixture with chloroflourocarbons (CFC's).

Another object of the present invention is to provide a photochemical reactor and process for the chlorination and/or oxidation of hydrochloroflourocarbons (HCFC's), hydroflourocarbons (HFC's) and hydrochlorocarbons (HCC's).

Another object of the present invention is to provide a photochemical reaction that is operable from a full vacuum to 20 atmospheres of pressure and operable from a temperature of minus −100° C. to 100° C.

Another object of the present invention is to provide a photochemical reactor that can be produced in an economical manner and is affordable by chemical manufacturers.

### SUMMARY OF THE INVENTION

In accordance with the present invention, there is provided a method of treatment of chemical impurities in used CFC-113 fluid using a photochemical reaction, wherein the chemical impurities are hydrogen-carbon bonded molecules, and the used CFC-113 fluid and the chemical impurities form an azeotropic or pseudo-azeotropic mixture, including the following steps of:
1) placing used CFC-113 fluid containing the chemical impurities into a photochemical reactor having a process compartment;
2) placing halogen fluid into said photochemical reactor, wherein the halogen fluid is selected from a group consisting of chlorine (Cl₂), bromine (Br₂) and iodine (I₂);
3) irradiating the used CFC-113 fluid and the halogen fluid using radiant energy from lamps in the visible and ultraviolet light regions of the electromagnetic spectrum to conduct thermolysis, photolysis and photochemical treatment;
4) halogenating the hydrogen-carbon bonded molecules in the chemical impurities with the halogen fluid to form halogenated chemical impurities during a dwell time period for elimination of the azeotropic mixture; and
5) removing the halogenated impurities by physical means, wherein the physical means include distillation, adsorption or extraction.

The present invention also provides for a photochemical reactor for transforming chemical impurities in used CFC fluids using a photochemical reaction; wherein the chemical impurities are molecules which contain hydrogen-carbon bonded molecules and the used CFC fluid and the chemical impurities form an azeotropic or pseudo-azeotropic mixture therein. The photochemical reactor includes a housing shell member; and the housing shell member has a cover member being attached thereto by a seal for forming a process compartment therein for receiving the used CFC fluid therein.

The photochemical reactor further includes a plurality of tube-lamp sleeves each having a tube retainer and seal member for sealing each of the tube-lamp sleeves within the cover member. Each of the tube-lamp sleeves are for holding a UV lamp therein, the UV lamps are used for irradiating the used CFC fluid and the halogen fluid by using radiant energy from the UV lamps in the visible and ultraviolet light regions of the electromagnetic spectrum in order to conduct thermolysis, photolysis and photochemical treatment of the used CFC fluid in the process compartment. The process compartment is used for halogenating the used CFC fluid for a pre-determined reaction period in order to transform the chemical impurities within the used CFC's in order to produce a high-quality re-processed CFC fluid.

### BRIEF DESCRIPTION OF THE DRAWINGS

Further object, features, and advantage of the present invention will become apparent upon the consideration of the following detailed description of the presently-preferred embodiment when taken in conjunction with the accompanying drawings; wherein:

FIG. 1 is a schematic representation of the photochemical reactor of the preferred embodiment of the present invention showing the major component parts of the reactor apparatus;

FIG. 2a is a schematic illustration of the photochemical reactor of the present invention showing a housing shell member having a tube-lamp sleeve with a central pitch configuration;

FIG. 2b is a schematic illustration of the photochemical reactor of the present invention showing the housing shell member having a plurality of tube-lamp sleeves with a triangular pitch configuration;

FIG. 2c is a schematic illustration of the photochemical reactor of the present invention showing the housing shell member having multiple tube-lamp sleeves with a square pitch configuration;

FIG. 3 is a schematic representation of the photochemical reactor of the present invention showing a tube retainer and seal member for holding the tube-lamp sleeve within a cover member; and

FIG. 4 is an enlarged exploded schematic representation of the photochemical reactor of the present invention showing a tube-lamp sleeve ferrule assembly for the tube retainer and seal member.

### DETAILED DESCRIPTION OF THE PREFERRED EMBODIMENT

The preferred embodiment of the present invention provides for a method of transforming chemical impurities in used CFC-113 fluid 12 using a shell and tube-lamp photochemical reactor 10. The used CFC-113 fluid 12 contains the contaminants or chemical impurities listed above. The molecules of the contaminants have a hydrogen-carbon bond. These contaminants may have any range of concentration in the used CFC fluid. The contaminant fluid and the used CFC-113 fluid may form an azeotropic or pseudo-azeotropic mixture. The photochemical reactor 10 is used to eliminate the chemical impurities in CFCs and saturated FCs. Also, it is used to provide for the production of high quality based products from HCFCs, HFCs, HCCs and HCs.

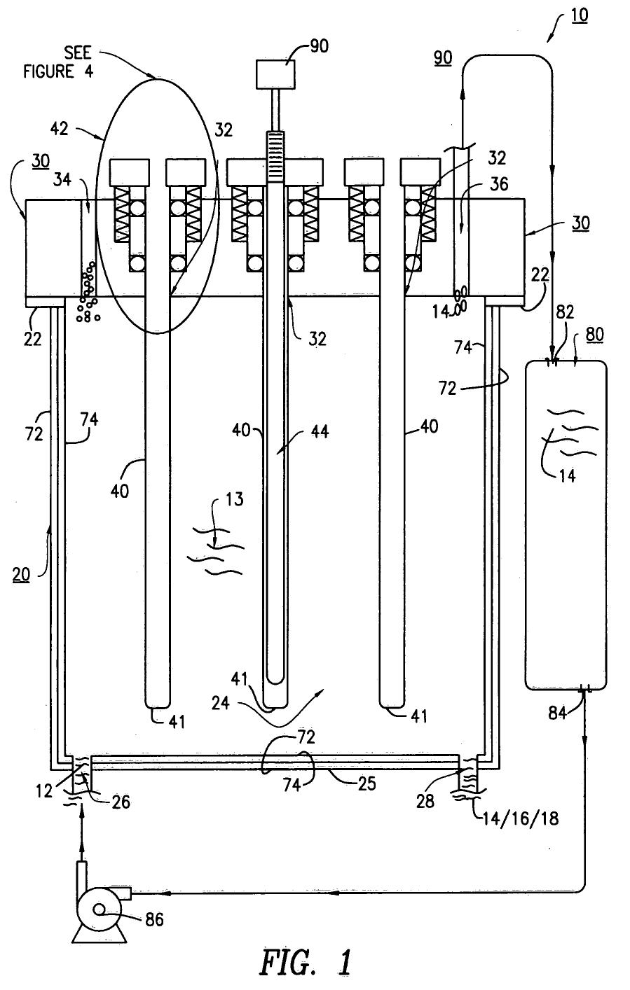

The apparatus of the shell and tube-lamp photochemical reactor 10, as shown in FIG. 1, provides the photochemical method of tranforming the chemical contaminant fluids for their easy removal by physical means from the used CFC-113 fluid 12 to meet a purity specification of 99.99% by eliminating the aforementioned total impurities (see above listing) so they have a hydrocarbon concentration of less than 1 ppm; a moisture content of less than 5 ppm; and have non-detectible solids therein. These purity specifications correspond to a virgin CFC-113 fluid.

The shell and tube-lamp photochemical reactor 10 is used for the photochemical treatment process for the halogenation or oxidation of the used CFC-113 fluid 12, such that the photochemical reactor 10 irradiates radiant energy using visible and ultraviolet wavelength light in the electromagnetic spectrum in order to halogenate or oxidize the chemical impurities of the used CFC-113 fluid 12 in order to yield the high-grade CFC-113. The photochemical reactor 10 is inert to the used CFC-113 fluid 12 being processed and is also inert to the halogen fluids 16 or oxygen fluids 18 used in the halogenation and/or oxygenation process of the contaminant fluid in the used CFC-113 fluid 12. The halogen fluid 16 is selected from a group consisting of chlorine (Cl₂), bromine (Br₂) and iodine (I₂). The operating conditions of the photochemical reactor 10 typically have an operating pressure in a range from a vacuum of 0.2 atmospheres absolute to 20 atmospheres, an operating temperature from minus −100° C. to +100° C. and an operating energy level in the electromagnetic spectrum region from 240 nm to 720 nm. The dwell time reaction is in a preferable, but not limited to, a range of 1 hour to 100 hours for the transforming the contaminants of the CFC-113 fluid 12 with the halogen gas 16 for yielding the high grade CFC-113 fluid 14.

The shell and tube-lamp photochemical reactor 10, as shown in FIGS. 1 through 4, includes a housing shell member 20 having a tube sheet member or cover member 30 thereon. The housing shell member 20 has an inside diameter in the range of 50 mm to 900 mm and has an overall length in the range of 300 mm to 3000 mm. The cover member 30 is attached to the housing shell member 20 with a tube sheet seal 22 for forming a process compartment 24 therein. Also, the process compartment 24 includes a bottom wall 25 having a liquid fluid loading port 26 therein and a liquid or gas fluid loading port 28 therein for the loading and unloading of the liquid or gas fluid, respectively, from the process compartment 24, as shown in FIG. 1 of the patent drawings. Further, the cover member 30 includes a vacuum, vent or pressure port 34 for introducing inert gas (nitrogen gas) into the process compartment 24. It is understood that port 34 also functions as a pressure/vent/vacuum port 34 for pressurization, evacuation or venting of gases from the process compartment 24, as depicted in FIG. 1. Additionally, the cover member 30 includes a return fluid port 36 for returning the fluid from the process compartment 24 to the inventory receiver tank 80, as shown in FIG. 1. The tube sheet member 30 also includes a plurality of tube-lamp sleeves 40 each having a tube-retainer and seal member 42 thereon for sealing each of the tube-lamp sleeves 40 within tube sheet member 30. The tube-lamp sleeve 40 is formed as a quartz tube having a domed end 41. The tube-lamp sleeve 40 has an outside diameter of 23 mm; an inside diameter of 20 mm; a wall thickness of 1.5 mm and a overall length of 1500 mm. Each of the tube-lamp sleeves 40 are for holding a UV lamp 44. The photochemical reactor 10 can use one or more UV lamps 44 depending upon the number of tube-lamp sleeves 40 used in the process compartment 24. The UV lamp 44 is a Phillips® UVC, 75 watts, soft glass TUV64T5. There is a clearance space between the quartz tube (tube-lamp sleeve) 40 and the housing shell member 20.

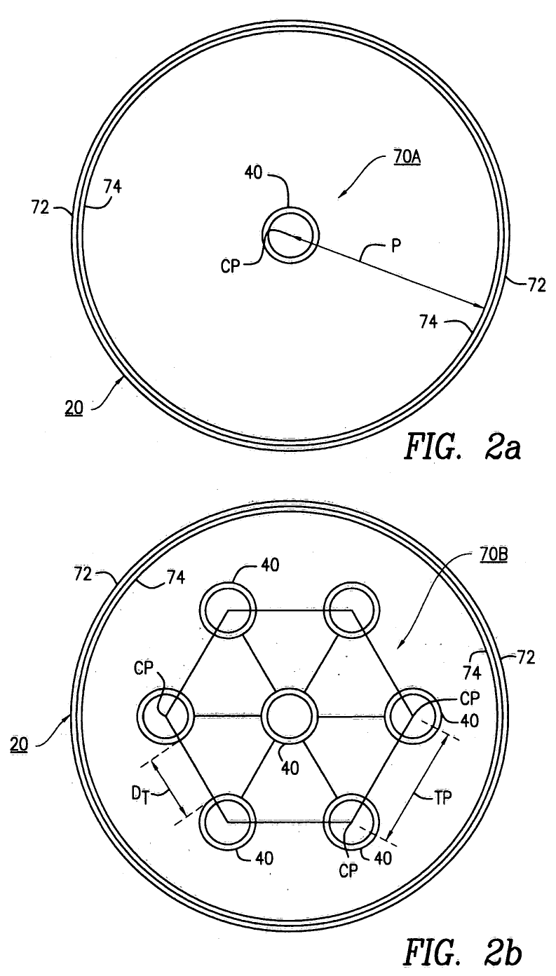

Further, each of the tube-lamp sleeves 40 can be configured in various tube pitch configurations, as shown in FIGS. 2a, 2b and 2c of the drawings, showing a central pitch configuration 70A, a triangular pitch configuration 70B and a square pitch configuration 70C, respectively. Pitch TP or SP is defined as the distance between the center point CP of adjacent tube-lamp sleeves 40, and pitch clearance DT or Ds is defined as the distance between the outer diameters of two adjacent tube-lamp sleeves 40, as depicted in FIGS. 2b and 2c of the drawings. The triangular pitch configuration 70B and the square pitch configuration 70C of the tube-lamp sleeves 40 are arranged in such a manner for optimizing the reaction time between the used CFC-113 fluid 12 and the halogen gas 16 in the process compartment 24. The photochemical reactor 10 further includes a power supply 90 for electrical power of the photochemical reactor 10.

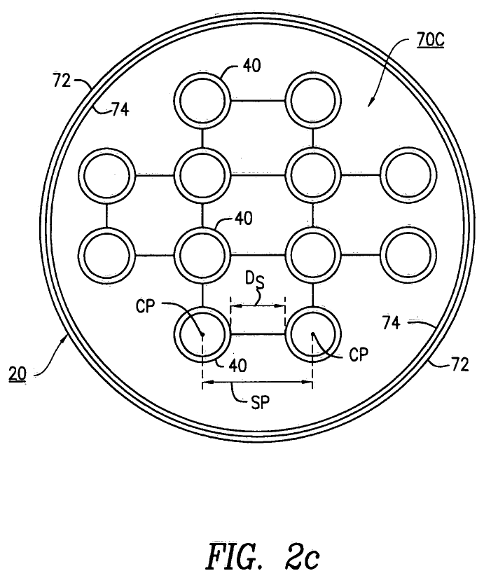

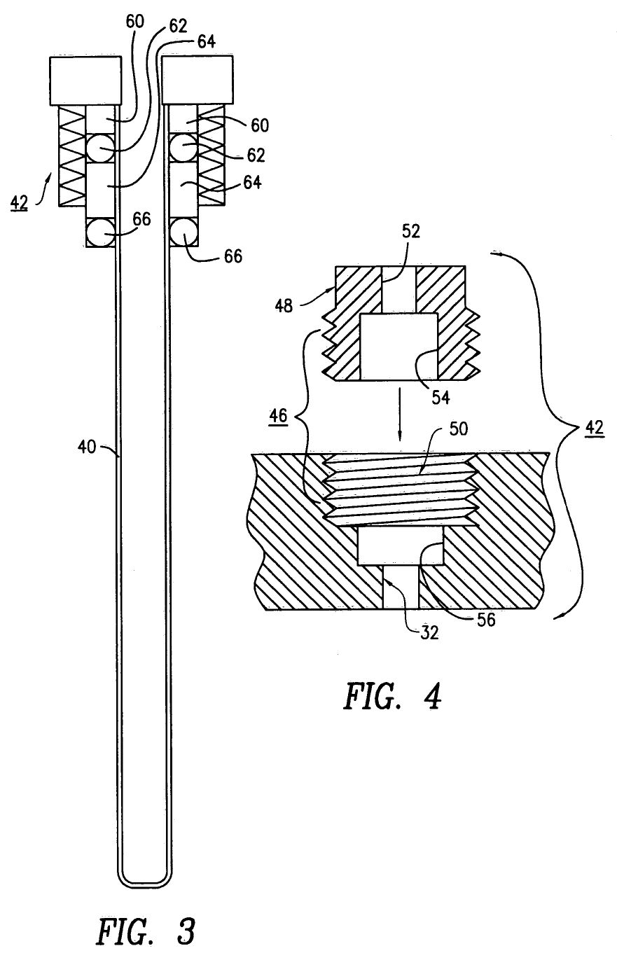

A tube hole opening 32 is drilled within the tube sheet member 30 with a slightly greater diameter than the outside diameter of the tube-lamp sleeve 40, in order to easily remove the tube-lamp sleeve 40 from the cover member 30. The tube retainer and seal member 42 includes a tube sleeve ferrule assembly 46 having a threaded male ferrule section 48 and a threaded female ferrule section 50 for receiving threaded male ferrule section 48 there through. The threaded male ferrule section 48 includes an upper bore opening 52 and a lower bore opening 54. The threaded female ferrule section 50 includes an upper bore opening 56 and a lower bore opening 32. The tube sleeve ferrule assembly 46 further includes a first compression tube sleeve 60, a first O-ring 62, a second compression tube sleeve 64 and a second O-ring 66. Components 60,62,64 and 66 are aligned with each other and are held within bore openings 54 and 56 of the male and female ferrule sections 48 and 50, respectively, as shown in FIGS. 3 and 4 of the drawings, for sealing of the tube-lamp sleeves 40 within the cover member 30 in order to prevent the leaking of the used CFC-113 fluid and the halogen gas 16 or oxygen gas 18 from the shell member 20 of the photochemical reactor 10.

The shell member 20 has an exterior wall surface 72 and an interior wall surface 74. The exterior wall surface 72 can be made of stainless steel, steel or suitable metal materials, depending upon if the exterior wall surface 72 is used for temperature control, such as cooling or heating. The temperature within the process compartment 24 of the shell member 20 is controlled at the desired temperature condition by means of cooling or heating coils, cooling and heating jackets or other heat transfer means on the exterior wall surface 72 of the shell member 20. The interior wall surface 74 which is in contact with the halogen gas 16 and the used CFC-113 fluid 12 can be made from glass quartz or fluoropolymers, such as THV (Tetrafluoroethylene hexapropylene vinylidine). Similarly, the tube-lamp sleeve 40 is made from glass quartz or fluoropolymer, such as THV.

The used CFC-113 fluid 12 is introduced into the process compartment 24 of the photochemical reactor 10 via fluid loading port 26, and chlorine gas (Cl₂) 16 is introduced into the process compartment 24 via the gas loading port 28. After a reaction time has been completed, the transformed contaminant fluid and the CFC-113 fluid are then transferred to the next process step via the drain port 28. If the used CFC-113 fluid 12 inventory is larger than the process compartment 24, an inventory receiver tank 80 is used, such that a circulation pump 86 is used to circulate the used CFC-113 fluid 12 between the process compartment 24 and the receiver tank 80 until all of the hydrogen atoms of the impurities are substituted by chlorine within the process compartment 24 of the photochemical reactor 10. The inventory receiver tank 80 includes an inlet port 82 and an outlet port 84 for receiving and discharging the CFC-113 fluid from the inventory receiver tank 80.

### EXAMPLE 1

The photochemical reactor 10 uses a single UV lamp 44 in the central pitch configuration 70A (See FIG. 2a). The photochemical reactor is arranged in a horizontal or prone position. The shell member 20 has an inside diameter of 53 mm, and has an overall length of 1600 mm. The single tube-lamp sleeve 40 has a 23 mm outside diameter and a 1500 mm length. The tube-lamp sleeve 40 is made of quartz and has a 1.5 mm wall thickness. The inside diameter of the tube-lamp sleeve 40 is 20 mm. The UV lamp 44 used is a Phillips UVC, 75 watts, soft glass TUV64T5. The interior wall surface 74 of the shell member 20 is quartz glass-lined.

The process compartment 24 is loaded with 2.0 Kgs of used CFC-113 fluid 12 with a 1% contaminant level of neo hexane, wherein the used CFC-113 fluid 12 and the chemical impurities at the same temperature. Next, the operator adds 60 grams of chlorine gas 16 through the gas receiving port 34 of the process compartment 24. The initial temperature of the CFC-113 fluid 12 in the photochemical reactor 10 is about 20° C. and at the end of the dwell time period the temperature is 40° C. The pressure in the photochemical reactor 10 is in the range of 1000 mm of Hg to 2000 mm of Hg. In the next step, the UV lamp 44 is used for a dwell time period of 6 hrs. The highest percentage of UV energy being used is in the range of 240 nm to 340 nm.

After the reaction has been completed, the reacted mixture of CFC-113 fluid 13 is transferred through standard process techniques of physical separation to yield a pure CFC-113 fluid. The result of the final product analysis is 99.99% CFC-113 purity and can be shown by gas chromatography (GC) using flame ionization detection (FID). The purified CFC-113 fluid 14 has less than 1 ppm of hydrocarbon fluid or other fluid with hydrogen in their molecules as indicated by the infrared spectrum analyzer (FI-IR) showing the spectrum wavelength in the region of 3100 to 2800 cm⁻¹.

### EXAMPLE 2

The photochemical reactor 10 uses seven (7) tube-lamp sleeves 40 each having a single UV lamp 44, therein. The photochemical reactor 10 is arranged in a horizontal position. The tube-lamp sleeves 40 are arranged in a triangular pitch configuration 70B (See FIG. 2b), and have a triangular pitch TP of 63 mm between each centerpoint CP of the tube-lamp sleeves 40 and have a clearance D_{T} of 40 mm between each tube-lamp sleeve 40. The shell member 20 has an inside diameter of 200 mm, and has an overall length of 1800 mm. Each of the tube-lamp sleeves 40 has a 23 mm outside diameter and a 1500 mm length. The tube-lamp sleeve 40 is made of quartz and has a 1.5 mm wall thickness. The inside diameter of the tube-lamp sleeve 40 is 20 mm. The UV lamp 44 used is a Phillips UVC, 75 watts, soft glass TUV64T5. The interior wall surface 74 of the shell member 20 is quartz glass-lined.

The process compartment 24 is loaded with 60 Kgs of used CFC-113 fluid 12 with a 0.5% contaminant level of neo hexane and a 0.5% contaminant level of dichlorethylene, wherein the used CFC-113 fluid 12 and the chemical impurities boil at the same temperature. This above mixture of used CFC-113 fluid 12 was previously distilled and the composition remains constant, which is an indication of an azeotropic mixture condition. Next, the operator adds 1.8 Kgs of chlorine gas 16 through the gas receiving port 34 of the process compartment 24. The initial temperature of the CFC-113 fluid 12 in the photochemical reactor 10 is about 20° C. and at the end of the dwell time period the temperature is 40° C. The pressure in the photochemical reactor 10 is in the range of 1000 mm of Hg to 2000 mm of Hg. In the next step, the UV lamp 44 is used for a dwell time period of 24 hrs, where the highest percentage of UV energy being used is in the range of 240 nm to 340 nm.

After the reaction has been completed, the reacted mixture of CFC-113 fluid 13 is transferred through standard process techniques of physical separation to yield a pure CFC-113 fluid. The result of the final product analysis is 99.99% CFC-113 purity and can be shown by gas chromatography (GC) using flame ionization detection (FID). The purified CFC-113 fluid has less than 1 ppm of hydrocarbon fluid or other fluid with hydrogen in their molecules as indicated by the infrared spectrum analyzer (FI-IR) showing the spectrum wavelength in the region of 3100 to 2800 cm⁻¹.

### EXAMPLE 3

The photochemical reactor 10 uses twelve (I₂) tube-lamp sleeves 40 each having a single UV lamp 44 therein. The photochemical reactor is arranged in a horizontal position. The tube-lamp sleeves 40 are arranged in a square pitch configuration 70C (See FIG. 2c) and have a square pitch SP of 63 mm between each center point CP of the tube-lamp sleeves 40 and have a clearance Ds of 40 mm between each tube-lamp sleeve 40. The shell member 20 has an inside diameter of 250 mm, and has an overall length of 1800 mm. Each of the tube-lamp sleeves 40 has a 23 mm outside diameter and a 1500 mm length. The tube-lamp sleeve 40 is made of quartz and has a 1.5 mm wall thickness. The inside diameter of the tube-lamp sleeve 40 is 20 mm. The UV lamp 44 used is a Phillips UVC, 75 watts, soft glass TUV64T5. The interior wall surface 74 of the shell member 20 is quartz glass-lined.

A mixture of used refrigerant fluids was prepared with the following composition:

| | |
|---|---|
| CFC-113 | 98.00% |
| CFC-124 | 0.05% |
| CFC-123 | 0.05% |
| Neo hexane | 0.05% |
| Ethylenedichloride | 0.05% |

The aforementioned mixture was distilled previously and the composition remains constant, which is an indication of an azeotropic mixture condition. The process compartment 24 is loaded with 100 Kgs of these used refrigerant fluids. Next, the operator adds 3.0 Kgs of chlorine gas 16 through the gas receiving port 34 of the process compartment 24. The initial temperature of the refrigerant fluids in the photochemical reactor 10 is about 20° C. and at the end of the dwell time period the temperature is 43° C. The pressure in the photochemical reactor 10 is in the range of 1000 mm of Hg. In the next step, the UV lamp 44 is used for a dwell time period of 24 hrs. The highest percentage of UV energy being used is in the range of 240 nm to 340 nm.

After the reaction has been completed, the reacted mixture of refrigerant fluids is transferred through standard process techniques of physical separation to yield a pure refrigerant fluid. The result of the final product analysis is 99.99% purity shown by gas chromatography (GC) using flame ionization detection (FID) for the pure refrigerant fluid. The purified refrigerant fluid has less than 1 ppm of hydrocarbon fluid or other fluid with hydrogen in their molecules as indicated by the infrared spectrum analyzer (Fl-IR) showing the spectrum wavelength in the region of 3100 to 2800 cm^{-1.}

### EXAMPLE 4

The photochemical reactor 10 uses twelve (I₂) tube-lamp sleeves 40 each having a single UV lamp 44 therein. The photochemical reactor is arranged in a horizontal position. The tube-lamp sleeves 40 are arranged in a square pitch configuration 70C (See FIG. 2c) and have a square pitch SP of 63 mm between each center point CP of the tube-lamp sleeves 40 and have a clearance Ds of 40 mm between each tube-lamp sleeve 40. The shell member 20 has an inside diameter of 250 mm, and has an overall length of 1800 mm. Each of the tube-lamp sleeves 40 has a 23 mm outside diameter and a 1500 mm length. The tube-lamp sleeve 40 is made of quartz and has a 1.5 mm wall thickness. The inside diameter of the tube-lamp sleeve 40 is 20 mm. The UV lamp 44 used is a Phillips UVC, 75 watts, soft glass TUV64T5. The interior wall surface 74 of the shell member 20 is quartz glass-lined.

A mixture of used refrigerant fluids was prepared with the following composition:

| | |
|---|---|
| CFC-113 | 98.00% |
| CFC-124 | 0.05% |
| CFC-123 | 0.05% |
| Neo hexane | 0.05% |
| Ethylenedichloride | 0.05% |

The aforementioned mixture was distilled previously and the composition remains constant, which is an indication of an azeotropic mixture condition. The process compartment 24 is loaded with 200 Kgs of these used refrigerant fluids with the chemical impurities. Next, the operator adds 6.0 Kgs of chlorine gas 16 through the gas receiving port 34 of the process compartment 24. The initial temperature of the refrigerant fluids in the photochemical reactor 10 is about 20° C. and at the end of the dwell time period the temperature is 43° C. The pressure in the photochemical reactor 10 is in the range of 1000 mm of Hg. In the next step, the UV lamp 44 is used for a dwell time period of 48 hrs. The highest percentage of UV energy being used is in the range of 240 nm to 340 nm. In addition, the inventory receiver tank 80 and process compartment 24 continually circulate the 200 Kgs of used refrigerant fluids via the circulation pump 86 during the 48 hour reaction dwell time period.

After the reaction has been completed, the transformed mixture of refrigerant fluids is transferred through standard process techniques of physical separation to a yield of pure refrigerant fluid. The result of the final product analysis is 99.99% purity can be shown by gas chromatography (GC) using flame ionization detection (FID) for the pure refrigerant fluid. The purified refrigerant fluid has less than 1 ppm of hydrocarbon fluid or other fluid with hydrogen in their molecules as indicated by the infrared spectrum analyzer (FI-IR) showing the spectrum wavelength in the region of 3100 to 2800 cm^{-1.}

### EXAMPLE 5

The photochemical reactor 10 uses twelve (I₂) tube-lamp sleeves 40 each having a single UV lamp 44 therein. The photochemical reactor is arranged in a vertical position. The tube-lamp sleeves 40 are arranged in a square pitch configuration 70C (See FIG. 2c) and have a square pitch SP of 63 mm between each center point CP of the tube-lamp sleeves 40 and have a clearance Ds of 40 mm between each tube-lamp sleeve 40. The shell member 20 has an inside diameter of 250 mm, and has an overall length of 1800 mm. Each of the tube-lamp sleeves 40 has a 23 mm outside diameter and a 1500 mm length. The tube-lamp sleeve 40 is made of quartz and has a 1.5 mm wall thickness. The inside diameter of the tube-lamp sleeve 40 is 20 mm. The UV lamp 44 used is a Phillips UVC, 75 watts, soft glass TUV64T5. The interior wall surface 74 of the shell member 20 is quartz glass-lined.

The process compartment 24 is loaded with 200 Kgs of used CFC-113 fluid 12 via loading port 26 with a 10.0% contaminant level of methylene chloride, wherein the used CFC-113 fluid 12 and the chemical impurities are boiling at the same temperature. This above mixture of used CFC-113 fluid 12 was distilled previously and the composition remains constant, which is an indication of an azeotropic mixture condition. The operator then adds dry air 18 via injection port 28 at a rate of 10 liters/minute to the mixture of used CFC-113 fluid. The initial temperature of the CFC-113 fluid 12 in the photochemical reactor 10 is about 20° C. and at the end of the dwell time period the temperature is 40° C. The pressure in the photochemical reactor 10 is in the range of 1000 mm of HG to 2000 mm of Hg. In the next step, the UV lamp 44 is used for a dwell time period of 48 hrs. The highest percentage of UV energy being used is in the range of 240 nm to 340 nm.

The methylene chloride is then oxidized and converted to carbon dioxide (CO₂) and hydrogen chloride (HCl). This gaseous reaction produces hydrogen chloride (from the oxidation of methylene chloride) and is passed to a caustic scrubber where the hydrogen chloride (HCl) is neutralized. The used CFC-113 fluid remains in the process compartment 24 until all of the methylene chloride is oxidized and the reacted mixture of CFC-113 fluid 13 is free of any methylene chloride. Then, the transformed mixture of CFC-113 fluid 13 is transferred through standard process techniques of physical separation to yield a pure CFC-113 fluid. The result of the final product analysis is 99.99% CFC-113 purity shown by gas chromatography (GC) using flame ionization detection (FID). The purified CFC-113 fluid 14 has less than 1 ppm of hydrocarbon fluid or other fluid with hydrogen in their molecules as indicated by the infrared spectrum analyzer (FI-IR) showing the spectrum wavelength in the region of 3100 to 2800 cm⁻¹.

### EXAMPLE 6

The photochemical reactor 10 uses seven (7) tube-lamp sleeves 40 each having a single UV lamp 44 therein. The photochemical reactor 10 is arranged in a vertical position. The tube-lamp sleeves 40 are arranged in a triangular pitch configuration 70B (See FIG. 2b), and have a triangular pitch TP of 63 mm between each centerpoint CP of the tube-lamp sleeves 40 and have a clearance DT of 40 mm between each tube-lamp sleeve 40. The shell member 20 has an inside diameter of 200 mm, and has an overall length of 1800 mm. Each of the tube-lamp sleeves 40 has a 23 mm outside diameter and 1500 mm length. The tube-lamp sleeve 40 is made of quartz and has a 1.5 mm wall thickness. The inside diameter of the tube-lamp sleeve 40 is 20 mm. The interior wall surface 74 of the shell member 20 is quartz glass-lined.

The process compartment 24 is loaded with 50 Kgs of an azeotropic mixture of 50% used dichlorodifluoromethene (CFC-12) and 50% used tetrafluoroethene (HFC-134a) wherein the azeotropic mixture boils at the same concentration. This above mixture of used CFC-12 and HFC-134a fluids were previously distilled and the composition remains constant, which is an indication of an azeotropic mixture condition. Next, the operator adds 20 Kgs of chlorine gas 16 through the gas receiving port 28 of the process compartment 24. The initial temperature of the used refrigerant fluids in the photochemical reactor 10 is about 20° C. and at the end of the dwell time period the temperature is 38° C. The pressure in the photochemical reactor 10 is in the range of 9 to 10 atmospheres. In the next step, the UV lamp 44 is used for a dwell time period of 24 hrs. The highest percentage of UV energy being used is in the range of 240 nm to 340 nm.

After the reaction has been completed the azeotropic condition disappears, the transformed HFC 134a mixture is converted to HCFC-124 and CFC-114. This mixture is transferred through standard process techniques of physical separation to yield pure refrigerant fluids. The result of the final product analysis is 99.99% purity can be shown by gas chromatography (GC) using flame ionization detection (FID).

### EXAMPLE 7

The photochemical reactor 10 uses a single UV lamp 44 in the central pitch configuration 70A (See FIG. 2a). The photochemical reactor is arranged in a horizontal or prone position. The shell member 20 has an inside diameter of 53 mm, and has an overall length of 1600 mm. The single tube-lamp sleeve 40 has a 23 mm outside diameter and a 1500 mm length. The tube-lamp sleeve 40 is made of quartz and has a 1.5 mm wall thickness. The inside diameter of the tube-lamp sleeve 40 is 20 mm. The UV lamp 44 used is a Phillips UVC, 75 watts, soft glass TUV64T5. The interior wall surface 74 of the shell member 20 is quartz glass-lined.

The process compartment 24 is loaded with 2.0 Kgs of used octafluorocyclebutane fluid with chemical impurities of hexafluorocyclebutene and hexafluoro-1-3-butadiene. Next, the operator adds 60 grams of chlorine gas 16 through the gas receiving port 34 of the process compartment 24. The initial temperature of the fluid 12 in the photochemical reactor 10 is about 20° C. and at the end of the dwell time period the temperature is 40° C. The pressure in the photochemical reactor 10 is in the range of 1000 mm of Hg to 2000 mm of Hg. In the next step, the UV lamp 44 is used for a dwell time period of 6 hrs. The highest percentage of UV energy being used is in the range of 240 nm to 340 nm, such that with the photolysis of the chlorine molecules, the chlorine radical produced induces that one of the two pairs of covalent electron bonds from the double bond chlorine-bond carbon is broken. This chemical reaction eliminates the azeotropic condition between the halogenated impurities and octafluorocyclobutane.

After the reaction has been completed, the reacted mixture of fluid is transferred through a standard process techniques of physical separation to yield a pure octafluorocyclebutane fluid. The result of the final product analysis is 99.99% purity of the octafuorocyclebutane fluid can be shown by gas chromatography (GC) using flame ionization detection (FID).

### EXAMPLE 8

The photochemical reactor 10 uses a single UV lamp 44 in the central pitch configuration 70A (See FIG. 2a). The photochemical reactor is arranged in a vertical position. The shell member 20 has an inside diameter of 53 mm, and has an overall length of 1600 mm. The single tube-lamp sleeve 40 has a 23 mm outside diameter and a 1500 mm length. The tube-lamp sleeve 40 is made of quartz and has a 1.5 mm wall thickness. The inside diameter of the tube-lamp sleeve 40 is 20 mm. The UV lamp 44 used is a Phillips UVC, 75 watts, soft glass TUV64T5. The interior wall surface 74 of the shell member 20 is quartz glass-lined.

The process compartment 24 is loaded with 2.0 Kgs of used HCFC-123 fluid. Next, the operator adds oxygen (O₂) gas 18 at a rate of 10 liters/min through the gas port 28 of the process compartment 24. The initial temperature of the HCFC-123 fluid in the photochemical reactor 10 is about 20° C. and at the end of the dwell time period the temperature is 40° C. The pressure in the photochemical reactor 10 is in the range of 1000 mm of Hg to 2000 mm of Hg. In the next step, the UV lamp 44 is used for a dwell time period of 8 hrs. The highest percentage of UV energy being used is in the range of 240 nm to 340 nm.

After the reaction has been completed, the HCFC-123 fluid is converted to trifluoro-acetyl chloride. Trifluoro-acetyl chloride can be extracted by standard process techniques of physical separation to yield pure trifluoro-acetyl chloride fluid. The result of the final product analysis is 99.99% trifluoro-acetyl chloride purity can be shown by gas chromatography (GC) using flame ionization detection (FID).

### Advantages of the Present Invention

Accordingly, an advantage of the present invention is that it provides for a photochemical reactor, in the form of a shell and tube-lamp reactor, such that the tube-lamp irradiates radiant energy of the visible and ultraviolet wave length light in the electromagnetic spectrum in order to halogenate or oxidize hydrochlorofluorocarbons (HCFCs), hydrofluorocarbons (HFCs), hydrochlorocarbons (HCCs), or hydrocarbons (HCs).

Another advantage of the present invention is that it provides for a photochemical reactor in order to halogenate or oxidize the chemical impurities present in the used CFCs or saturated FCs in the form of an azeotropic or pseudo-azeotropic mixture, such that the photochemical reaction transforms the chemical impurities which then changes the physical and chemical properties of the mixtures of the CFCs or FCs and all of the azeotropic conditions disappear.

Another advantage of the present invention is that it provides for a photochemical reactor that uses radiant tube-lamps for the process of irradiation of radiating heat and energy using visible and ultraviolet light in order to promote the thermolysis and photolysis of molecules, such as chlorine (Cl₂) and oxygen (O₂) molecules.

Another advantage of the present invention is that it provides for a photochemical reactor and process that is capable of transforming fluids such as hydrochlorofluorocarbons (HCFCs), hydrofluorocarbons (HFCs), hydrochlorocarbons (HCCs), and hydrocarbons (HCs).

Another advantage of the present invention is that it provides for a photochemical reaction that is operable from a full vacuum to 20 atmospheres of pressure and operable from a temperature of minus −100° C. to 100° C.

A further advantage of the present invention is that it provides for a photochemical reactor that can be produced in an economical manner and is affordable by chemical manufacturers.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

1. A method of treatment of chemical impurities in used CFC-113 fluid using a photochemical reaction, wherein the chemical impurities are molecules that have hydrogen atoms in the hydrogen-carbon bonds, and the used CFC-113 fluid and the chemical impurities form an azeotropic or pseudoazeotropic mixture, comprising the steps of:
a) placing used CFC-113 fluid containing the chemical impurities into a photochemical reactor having a process compartment;
b) placing halogen fluid into said photochemical reactor;
c) irradiating said used CFC-113 fluid and said halogen fluid using radiant energy from lamps in the visible and ultraviolet light regions of the electromagnetic spectrum to conduct thermolysis, photolysis and photochemical treatment;
d) halogenating said hydrogen-carbon bonded molecules in said chemical impurities with said halogen fluid to form halogenated chemical impurities during a dwell time period for elimination of said azeotropic mixture; and
e) removing said halogenated impurities by physical means, wherein said physical means include the standard process techniques of physical separation.

2. A method of treatment of chemical impurities in accordance with claim 1, further including the step of:
a) processing said used CFC-113 fluid in said photochemical reactor at an operating pressure in the range from a vacuum of 0.1 atmosphere absolute to 20 atmospheres, at an operating temperature from −100° C. to +100° C. and at an operating radiant energy level in the region of the electromagnetic spectrum from 240 nm to 720 nm, wherein said halogen fluid is chlorine (Cl₂).

3. A method of treatment of chemical impurities in accordance with claim 1, further including the step of:
a) pumping said used CFC-113 fluid from an inventory receiver tank to said process compartment of said photochemical reactor, such that a circulation pump is used to circulate said used CFC-113 fluid between said process compartment and said receiver tank until all of said hydrogen atoms of said hydrogen-carbon bonds of said molecules in said chemical impurities are substituted by said halogen fluid within said process compartment of said photochemical reactor.

4. A method of treatment of chemical impurities in accordance with claim 3, further including the step of:
a) reacting said impurities of the used CFC-113 fluid in said process compartment for said dwell time period is in the range of 1 hour to 100 hours, depending upon the concentration of said chemical impurities of said used CFC-113 fluid.

5. A method of treatment of chemical impurities in used chlorofluorocarbon (CFC) fluid using a photochemical reaction, wherein the chemical impurities are molecules that have hydrogen atoms in the hydrogen-carbon bonds, and the used CFC fluid and the chemical impurities form an azeotropic or pseudoazeotropic mixture, comprising the steps of:
a) placing used CFC fluid containing the chemical impurities into a photochemical reactor having a process compartment;
b) placing halogen fluid into said photochemical reactor, wherein said halogen fluid is selected from a group consisting of chlorine (Cl₂), bromine (Br₂) and iodine (I₂);
c) irradiating said used CFC fluid and said halogen gas using radiant energy from lamps in the visible and ultraviolet light regions of the electromagnetic spectrum to conduct thermolysis, photolysis and photochemical treatment;
d) halogenating said hydrogen-carbon bonds of said molecules in said chemical impurities with a halogen gas to form halogenated chemical impurities during a dwell time period for elimination of said azeotropic mixture; and
e) removing said halogenated impurities by physical means, wherein said physical means include standard process techniques of physical separation.

6. A method of treatment of chemical impurities in used fluorocarbon (FC) fluid using a photochemical reaction, wherein the chemical impurities are molecules which contain one or more double bonds, and the used FC fluid and the chemical impurities form an azeotropic or pseudoazeotropic mixture, comprising the steps of:
a) placing used FC fluid containing the chemical impurities into a photochemical reactor having a process compartment;
b) placing halogen fluid into said photochemical reactor, wherein said halogen fluid is selected from a group consisting of chlorine (Cl₂), bromine (Br₂) and iodine (I₂);
c) irradiating said used FC fluid and said halogen fluid using radiant energy from lamps in the visible and ultraviolet light regions of the electromagnetic spectrum to conduct thermolysis, photolysis and photochemical treatment;
d) halogenating said double bonds of said molecules in said chemical impurities with said halogen fluid to form halogenated chemical impurities during a dwell time period for elimination of said azeotropic mixture; and
e) removing said halogenated impurities by physical means, wherein said physical means include standard process techniques of physical separation.

7. A method of treatment of chemical impurities in used CFC-113 fluid using a photochemical reaction, wherein the chemical impurities are molecules which contain a hydrogen atom and a halogen atom on the same carbon of the molecule, and the used CFC-113 fluid and the chemical impurities form an azeotropic or pseudo-azeotropic mixture, comprising the steps of:
a) placing the used CFC-113 fluid containing chemical impurities into a photochemical reactor having a process compartment;
b) placing oxygen (O₂) fluid or air into said photochemical reactor;
c) irradiating said used CFC-113 fluid and said oxygen fluid or air using radiant energy from lamps in the visible and ultraviolet regions of the electromagnetic spectrum to conduct thermolysis, photolysis and photochemical treatment;
d) reacting the hydrogen atom and halogen atom of said molecules of said chemical impurities with said oxygen (O₂) fluid or air by oxygenation to form oxidized chemical impurities during a dwell time period for the elimination of said azeotropic mixture; and
e) removing said oxidized chemical impurities from said used CFC-113 fluid by physical means, wherein said physical means include standard process techniques of physical separation.

8. A method of treatment of chemical impurities in accordance with claim 7, further including the step of:
a) processing said used CFC-113 fluid in said photochemical reactor at an operating pressure in the range from a vacuum of 1 mmHg to 20 atmospheres, at an operating temperature from −100° C. to +100° C. and at an operating radiant energy level in the region of the electromagnetic spectrum from 240 nm to 720 nm.

9. A method of treatment of chemical impurities in accordance with claim 7, further including the step of:
a) pumping said used CFC-113 fluid from an inventory receiver tank to said process compartment of said photochemical reactor, such that a circulation pump is used to circulate said used CFC-113 fluid between said process compartment and said receiver tank until all of said hydrogen atoms and said chlorine atoms are substituted by said oxygen fluid within said process compartment of said photochemical reactor.

10. A method of treatment of chemical impurities in accordance with claim 9, further including the step of:
a) reacting said used CFC-113 fluid in said process compartment for said dwell time period in the range of 1 hour to 100 hours, depending upon the concentration of said chemical impurities of said used CFC-113 fluid.

11. A method of treatment of chemical impurities in used chlorofluorocarbon (CFC) fluid using a photochemical reaction, wherein the chemical impurities are molecules which contain a hydrogen atom and a halogen atom on the same carbon of the molecule, and the used CFC fluid and the chemical impurities form an azeotropic or pseudoazeotropic mixture, comprising the steps of:
a) placing the used CFC fluid containing chemical impurities into a photochemical reactor having a process compartment;
b) placing oxygen (O₂) fluid or air into said photochemical reactor;
c) irradiating said used CFC fluid and said oxygen fluid or air using radiant energy from lamps in the visible and ultraviolet regions of the electromagnetic spectrum to conduct thermolysis, photolysis and photochemical treatment;
d) reacting the hydrogen atom and halogen atom of said molecules of said chemical impurities with said oxygen (O₂) fluid or air by oxygenation to form oxidized chemical impurities during a dwell time period for the elimination of said azeotropic mixture; and
e) removing said oxidized chemical impurities from said used CFC fluid by physical means, wherein said physical means include standard process techniques of physical separation.

12. A method of treating hydrochlorofluorocarbon (HCFC) fluids using a photochemical reaction, wherein the HCFC molecules contain a hydrogen atom and a halogen atom on the same carbon of the HCFC molecule, comprising the steps of:
a) placing said HCFC fluid into a photochemical reactor having a process compartment;
b) placing oxygen (O₂) fluid or air into said photochemical reactor;
c) irradiating said HCFC fluid and said oxygen fluid or air using radiant energy from lamps in the visible and ultraviolet regions of the electromagnetic spectrum to conduct thermolysis, photolysis and photochemical treatment;
d) reacting the hydrogen atom and halogen atom of said molecules of said HCFC fluid with said oxygen (O₂) fluid or air by oxygenation to form an acetyl fluid during a dwell time period; and
e) removing said acetyl fluid from said HCFC fluid by standard process techniques of physical separation.

13. A method of treating hydrofluorocarbon (HFC) fluids using a photochemical reactor, wherein the HFC molecules contain a hydrogen atom and a halogen atom on the same carbon of the HFC molecule, comprising the steps of:
a) placing said HFC fluid into a photochemical reactor having a process compartment;
b) placing oxygen fluid or air into said photochemical reactor;
c) irradiating said HFC fluid and said oxygen fluid or air using radiant energy from lamps in the visible and ultraviolet region of the electromagnetic spectrum to conduct thermolysis, photolysis and photochemical treatment;
d) reacting by methatesis of oxygen by substitution of an atom of hydrogen and an atom of flourine from the same carbon with oxygen fluid and thereby forming an acetyl fluid; and
e) removing said fluid acetyl by standard techniques of physical separation such as distillation and adsorption.

14. A photochemical reactor for transforming a reactant fluid by employing a photochemical reaction wherein the reactant fluid has molecules which contain hydrogen-carbon bonds which form an azeotropic or pseudoazeotropic mixture therein, comprising:
a) a photochemical reactor having a housing shell member;
b) said housing shell member having a cover member being attached thereto by a seal for forming a process compartment therein for receiving the reactant fluid therein;
c) a plurality of tube-lamp sleeves each having a tube retainer and seal member for sealing each of said tube-lamp sleeves within said cover member;
d) each of said tube-lamp sleeves for holding a UV lamp therein, said UV lamps for irradiating the reactant fluid and a halogen gas or oxygen gas, and using radiant energy from said UV lamps in the visible and ultraviolet light regions of the electromagnetic spectrum in order to conduct thermolysis, photolysis and photochemical treatment of the reactant fluid in said process compartment; and
e) said process compartment for halogenating or oxidizing the reactant fluid for a pre-determined dwell reaction period in order to transform the reactant fluid in order to produce a high-quality product.

15. A photochemical reactor in accordance with claim 14, further including an inventory receiver tank having a circulation pump, such that said circulation pump is used to circulate the reactant fluid between said process compartment and said receiver tank until all of said hydrogen-carbon bonds are substituted by the halogen gas within said process compartment of said photochemical reactor.

16. A photochemical reactor in accordance with claim 14, wherein said housing shell member includes an exterior wall and an interior wall in contact with each other.

17. A photochemical reactor in accordance with claim 16, wherein said housing shell member includes heat transfer means for conducting the transfer of heat or cold on said exterior wall.

18. A photochemical reactor in accordance with claim 17, wherein said exterior wall is made from stainless steel, steel or other suitable metal materials for conducting the transfer of heat or cold by said heat transfer means.

19. A photochemical reactor in accordance with claim 17, wherein said heat transfer means include heating or cooling jackets on said exterior wall of said housing shell member.

20. A photochemical reactor in accordance with claim 17, wherein said heat transfer means include heating or cooling coils on said exterior wall of said housing shell member.

21. A photochemical reactor in accordance with claim 17, wherein said heat transfer means for conducting the transfer of heat or cold on said exterior wall has a temperature range from −100° C. to +100° C.

22. A photochemical reactor in accordance with claim 16, wherein said interior wall is made from glass quartz or fluoropolymers for allowing unreacted/inert contact with said halogen fluid and said reactant fluids.

23. A photochemical reactor in accordance with claim 20, wherein said fluoropolymer is tetrafluoroethylene hexapropylene vinylidine (THV).

24. A photochemical reactor in accordance with claim 14, wherein said housing shell member has a fluid loading port therein and has a fluid drain port therein for loading and is unloading the reactant fluids, respectively, into and from said process compartment.

25. A photochemical reactor in accordance with claim 14, wherein said housing shell member has an inside diameter in the range of 5 cm to 100 cm and an overall length in the range of 10 cm to 300 cm.

26. A photochemical reactor in accordance with claim 14, wherein said cover member includes a gas receiving port for introducing a halogen fluid or other fluids into said process compartment.

27. A photochemical reactor in accordance with claim 14, wherein said cover member includes a pressure port for pressurization of said process compartment at a operating pressure level in a range of from a vacuum of 1 mm Hg to 20 atmospheres.

28. A photochemical reactor in accordance with claim 14, wherein said halogen fluid is selected from the group consisting of chlorine (Cl₂), bromine (Br₂), and iodine (I₂).

29. A photochemical reactor in accordance with claim 14, wherein said cover member includes one or more hole openings for receiving one or more of said tube-lamp sleeves therethrough.

30. A photochemical reactor in accordance with claim 14, wherein said tube-lamp sleeve is formed as a quartz glass tube having a dome end, said tube-lamp sleeve having an outside diameter range of 10 mm to 40 mm, an inside diameter range of 8 mm to 38 mm, a wall thickness range of 0.5 mm to 5 mm, and an overall length in the range of 10 cm to 300 cm.

31. A photochemical reactor in accordance with claim 14, wherein said tube retainer and seal member includes a tube sleeve ferrule assembly having a threaded male ferrule section and a threaded female ferrule section for receiving said threaded male ferrule section therein.

32. A photochemical reactor in accordance with claim 31, wherein said male and female ferrule sections cooperate for receiving a plurality of O-rings for sealing of said tube-lamp sleeve within said cover member in order to prevent leaking of the reactant fluid and the halogen and/or oxygen fluid from said housing shell member.

33. A photochemical reactor in accordance with claim 14, wherein said tube-lamp sleeves are arranged in a triangular pitch configuration within said housing shell member for optimizing the reaction time of the reactant fluid and the oxygen and/or halogen fluid in said process compartment.

34. A photochemical reactor in accordance with claim 14, wherein said tube-lamp sleeves are arranged in a square pitch configuration within said housing shell member for optimizing the reaction time of the reactant fluid and the oxygen and/or halogen fluid in said process compartment.

35. A photochemical reactor in accordance with claim 14, wherein said UV lamp operates at a radiant energy level in the electromagnetic spectrum region in the range from 240 mm to 720 mm.

36. A photochemical reactor in accordance with claim 16, wherein said dwell reaction period is in the range of 1 hour to 100 hours for reacting the reactant fluid with the oxygen and/or halogen fluid in order to yield a 99.99% purity product fluid.
